(19) United States
(12) Patent Application Publication
(10) Pub. No.: US 2023/0271130 A1
(43) Pub. Date: Aug. 31, 2023

(54) A FACILITY AND A MEMBRANE PROCESS FOR SEPARATING METHANE AND CARBON DIOXIDE FROM A GAS STREAM

(71) Applicant: EVONIK OPERATIONS GMBH, Essen (DE)

(72) Inventors: Markus PRISKE, Salzburg (AT); Hanns KUHLMANN, Witten (DE); Georg Friedrich THIELE, Friedberg (DE)

(73) Assignee: EVONIK OPERATIONS GMBH, Essen (DE)

(21) Appl. No.: 18/015,866

(22) PCT Filed: Jul. 1, 2021
(86) PCT No.: PCT/EP2021/068142

§ 371 Date: Jan. 12, 2023

(30) Foreign Application Priority Data
Jul. 14, 2020 (EP) 20185597.0

(51) Int. Cl. B01D 53/22; B01D 53/30

(52) U.S. Cl. CPC B01D 53/227; B01D 53/228; B01D 53/30; B01D 2053/224; B01D 2256/245; B01D 2257/504

(57) ABSTRACT

A facility and a process with four membrane separation units, where the second separation unit separates the retentate of the first unit, the third separation unit separates the permeate of the first unit, the fourth separation unit separates the retentate of the third unit, the permeate of the second unit and the retentate of the fourth unit are recycled to the feed to the first unit, the permeate of the fourth unit is passed to a methane oxidation unit and the permeate of the third unit is discharged to the atmosphere allows separating methane and carbon dioxide from a gas stream, providing a methane rich stream with the retentate of the second unit at a high methane yield and adhering to low limits for methane discharge to the atmosphere with a small size methane oxidation unit.

### FIELD OF THE INVENTION

The invention is directed at a membrane process and a facility for separating methane and carbon dioxide from a gas stream, providing a methane stream suitable for injection into a natural gas grid, which can achieve low emission of methane to the atmosphere with little extra equipment and energy consumption.

### BACKGROUND OF THE INVENTION

Biogas resulting from anaerobic fermentation, such as biogas from an anaerobic digester or a landfill gas, comprises methane and carbon dioxide as the major components. Separating methane from biogas in a quality suitable for feeding the methane into a gas distribution grid is of commercial interest. Membrane processes are advantageous for separating methane from carbon dioxide as they do not require an absorbent for carbon dioxide and can be operated with low energy consumption. Since methane is a more potent greenhouse gas than carbon dioxide, the carbon dioxide enriched stream obtained by a membrane separation process can only be discharged to the atmosphere if it is separated with a low methane content or subjected to an additional treatment for methane removal. Such additional treatment for methane removal consumes energy and requires extra equipment.

WO 2012/000727 discloses a membrane process with three membrane units which can separate biogas into a biomethane stream containing more than 98 vol-% methane and a carbon dioxide enriched stream containing about 0.5% methane at a low recycle rate of less than 60% which makes the process energy efficient.

WO 2015/036709 discloses a membrane process with four membrane units which aims at further reducing the energy required for compressing recycled gas but provides a lower methane recovery compared to the process of WO 2012/000727. The process provides two carbon dioxide enriched streams from the third and the fourth membrane unit. WO 2015/036709 suggests that these two streams may be separately or jointly treated by thermal oxidation, used for upgrading the carbon dioxide or discharged to the atmosphere.

At Sep. 24, 2018 the Oil and Gas Climate Initiative (OGCI) published a first methane emission target for its member companies. A base line for methane that gets lost when producing oil and gas of max. 0.32% and a target of 0.25% methane loss for 2025 was set.

Tightened regulations on emission of greenhouse gases, e.g. § 36 of the German “42. Verordnung über den Zugang zu Gasversorgungsnetzen (Gasnetzzugangsverordnung-GasNZV)”, require even more ambitious targets for lowering methane emissions from biogas upgrading or natural gas purification (max. 0.2%). The prior art membrane processes can achieve such goals only by significantly high recycle rates or by an additional step of removing methane from the carbon dioxide enriched streams before discharge to the atmosphere. Both measures increase costs and decrease efficiency of the prior art processes.

Therefore, a strong need remains for an efficient process for separating methane and carbon dioxide from a gas stream, which fulfills the requirement of the tightened regulations on emissions of greenhouse gases with little extra equipment and energy consumption.

Subject of the present invention was to provide a new facility and a new process having the disadvantages of the prior art processes and facilities to a reduced degree respectively not having the disadvantages of the prior art processes and facilities.

A specific problem of the present invention was to provide a new facility and a new process for separating methane and carbon dioxide from a gas stream, which fulfills the requirements of tightened regulations on emissions of greenhouse gases, in particular with regard to gas streams that are discharged to the atmosphere and that should have a methane content of below or equal to 0.3% by volume, preferably below or equal to 0.2% by volume.

Another specific problem of the present invention was to provide a new facility and a new process for separating methane and carbon dioxide from a gas stream, wherein at least one carbon dioxide enriched stream, that is discharged to the atmosphere, is provided having a methane content of below or equal to 0.3% by volume, preferably 0.2% by volume, without oxidative, methan removing post treatment step.

In another specific problem of the present invention a new facility and a new process for upgrading a gas comprising methane and carbon dioxide shall be provided, wherein a methane product stream having a methane content of more than or equal to 97% by volume can be obtained and simultaneously a methane yield higher than disclosed in WO 2015/036709 A1 can be achieved.

In another specific problem of the present invention a new facility and a new process for upgrading a gas comprising methane and carbon dioxide shall be provided, which are highly efficient in view of operating costs and/or invest costs. Preferably the invest and/or operating costs for gas recompression and/or post treatment of off-gas streams to reduce the methane content shall be minimized.

In another specific problem of the present invention a new facility and a new process for upgrading a gas comprising methane and carbon dioxide shall be provided, allowing to continuously fulfill regulatory requirements with regard to methane emission to the atmosphere even if the composition and/or flow rate of the raw gas stream vary.

Further problems solved by the present invention but not described before, can be derived from the subsequent description, examples, figures and claims.

### SUMMARY OF THE INVENTION

The inventor of the present invention has now surprisingly found that the problems described above, can be solved by using a membrane separation facility with four membrane units as known from WO 2015/036709, which facility has been modified by
a. connecting only the permeate outlet of the fourth membrane unit to a methane oxidation unit and discharging the permeate from the third membrane unit directly to the atmosphere,
b. configuring and operating the facility to provide a carbon dioxide concentration in the first permeate stream of from 90 to 99% by volume,
c. using membranes with a pure gas selectivity for carbon dioxide over methane of at least 30, determined at 20° C. and 5 bar, in the first membrane separation unit.

The facility and the process of the invention allow for adhering to strict regulatory requirements for methane emission to the atmosphere for both, the third and fourth permeate stream, even if the third permeate stream is not subjected to methane removing post treatment and directly discharged to the atmosphere. As shown in Comparative Examples 1a and 1 b below, the process of WO 2015/036709 A1, does not disclose any facility or process wherein a third permeate stream with a methane content of 0.3 Vol. % is provided without oxidative post treatment.

The achievement to provide a third permeate stream with a methane content of 0.3 Vol. % or below after the membrane separation allows to reduce invest costs for equipment for oxidative methane removal in the facility and process of the invention. Also, the operating costs for methane removal could be reduced compared to the prior art. In preferred embodiments of the invention it was in addition achieved to minimize the volume flow of the fourth permeate stream, which enables to further reduce the capacities for oxidative post-treatment and to further reduce invest and operating costs.

Compared to prior art processes the facility and process of the invention can be operated at with minimum cost for recompression even though tightened requirements for methane emission to the atmosphere are fulfilled.

Preferably the facility and process of the invention comprise means for direct or indirect measurement and/or means for controlling the methane concentration in the third permeate stream. In preferred embodiments the operating conditions of the first membrane unit of the facility are adjusted based on direct or indirect measuring the methane concentration in the third permeate stream. This allows to continuously provide a third permeate stream having a methane concentration of 0.3 Vol % or below even if the composition and/or flow rate of the raw gas stream change. Facility and process of the invention can therefore be used flexibly for different raw gas sources and raw gas sources with varying amounts and/or composition of the raw gas.

Process and facility of the invention provide methane product stream having very high methane contents and very high methane yield.

Further advantages of the facility and the process of the invention are revealed in the subsequent description, examples, figures and claims.

Subject of the invention is therefore a facility for separating methane and carbon dioxide from a gas stream, which facility comprises
a compressor (1);
four membrane separation units (2) to (5), each membrane separation unit comprising a gas separation membrane having higher permeance for carbon dioxide than for methane, a gas inlet, a retentate outlet and a permeate outlet;
a methane oxidation unit (6);
a raw gas conduit (7) connected to an inlet of the compressor (1);
a feed conduit (8) connecting an outlet of the compressor (1) with the gas inlet of the first membrane separation unit (2);
a first retentate conduit (9) connecting the retentate outlet of the first membrane separation unit (2) to the gas inlet of the second membrane separation unit (3);
a second retentate conduit (10) connected to the retentate outlet of the second membrane separation unit (3);
a first permeate conduit (11) connecting the permeate outlet of the first membrane separation unit (2) to the gas inlet of the third membrane separation unit (4);
a third retentate conduit (12) connecting the retentate outlet of the third membrane separation unit (4) to the gas inlet of the fourth membrane separation unit (5);
a fourth retentate conduit (13) connecting the retentate outlet of the fourth membrane separation unit (5) to an inlet of the compressor (1);
a second permeate conduit (14) connecting the permeate outlet of the second membrane separation unit (3) to an inlet of the compressor (1);
a third permeate conduit (15) connected to the permeate outlet of the third membrane separation unit (4); and
a fourth permeate conduit (16) connected to the permeate outlet of the fourth membrane separation unit (5)
characterized in that
the third permeate conduit (15) is configured to discharge the third permeate to the surrounding atmosphere;
the fourth permeate conduit (16) connects the permeate outlet of the fourth membrane separation unit (5) to the methane oxidation unit (6);
the first membrane separation unit (2) comprises a membrane with a with a pure gas selectivity for carbon dioxide over methane, determined at 20° C. and 5 bar, of at least 30, preferably of from 40 to 120 and more preferably of from 50 to 100;
the facility is configured to provide a carbon dioxide concentration in the gas stream in the first permeate conduit (11), the first permeate stream, in a range of from 90 to 99% by volume.

A further subject of the invention is a membrane process for separating methane and carbon dioxide from a gas stream, which process comprises
a) providing a facility of the invention;
b) introducing a raw gas stream, containing from 20 to 60% by volume, preferably 20 to 50% by volume, carbon dioxide and having a combined content of methane and carbon dioxide of at least 95% by volume, into the raw gas conduit (7) of said facility;
c) compressing the raw gas stream combined with recycle streams from the fourth retentate conduit (13) and the second permeate conduit (14) with compressor (1) to provide a feed stream at a feed pressure of from 7 to 25 bar and a temperature of from 15 to 50° C.;
d) separating the feed stream in the first membrane separation unit (2) into a first permeate stream and a first retentate stream, using a membrane with a mixed gas selectivity for carbon dioxide over methane of at least 30, preferably of from 40 to 100, at the feed pressure and the temperature of the feed stream, and selecting permeate side pressure in the first membrane separation unit and separation capacities in the four membrane separation units to provide a carbon dioxide concentration in the first permeate stream of from 90 to 99% by volume, the separation capacity of a membrane separation unit being the product of the membrane area and the membrane permeance for carbon dioxide at a temperature of 25° C. and a feed side pressure of 5 bar;
e) separating the first retentate stream in the second membrane separation unit (3) into a second retentate stream and a second permeate stream, further processing the second retentate stream or withdrawing the second retentate stream as a methane rich product stream and recycling the second permeate stream through the second permeate conduit (14);
f) separating the first permeate stream in the third membrane separation unit (4) into a third retentate stream and a third permeate stream, discharging the third permeate stream to the surrounding atmosphere without further methane removal;
g) separating the third retentate stream in the fourth membrane separation unit (5) into a fourth retentate stream and a fourth permeate stream, recycling the fourth retentate stream through the retentate conduit (13); and
h) oxidizing the fourth permeate stream in the methane oxidation unit (6) to provide an off-gas stream containing less than 0.3% by volume methane, which off-gas stream is discharged to the surrounding atmosphere.

### BRIEF DESCRIPTION OF DRAWINGS

FIG. 1 shows an embodiment of the facility of the invention where a methane concentration sensor (18) connected to the third permeate conduit (15) controls a pressure regulating valve (17) arranged in the fourth retentate conduit (13).

FIG. 2 shows an embodiment of the facility of the invention where methane concentration sensor (18) controls a flow regulating valve (20) in a conduit passing a heating or cooling fluid to a heat exchanger (19) in the feed conduit (8).

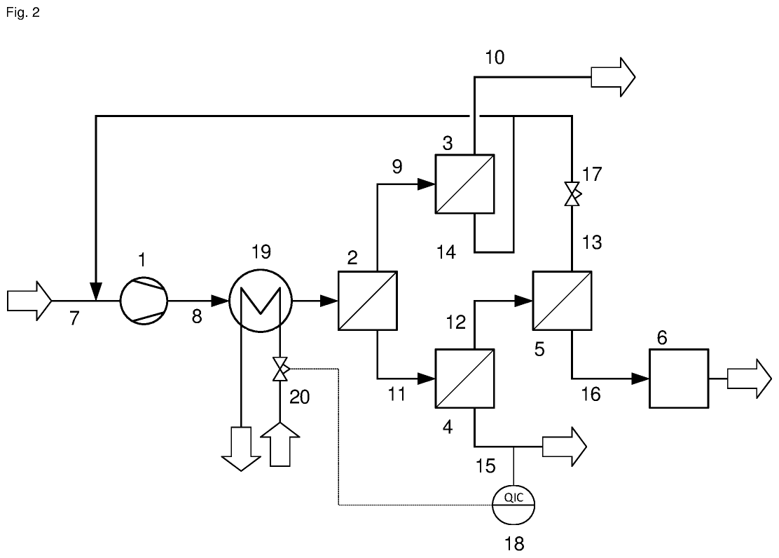

FIG. 3 shows an embodiment of the facility of the invention where the first membrane separation unit (2) comprises an additional permeate outlet and methane concentration sensor (18) controls a flow regulating valve (22) arranged in an additional conduit (21) connecting the additional permeate outlet with the gas inlet of the fourth membrane separation unit (5).

### DETAILED DESCRIPTION OF THE INVENTION

The facility of the invention for separating methane and carbon dioxide from a gas stream comprises a compressor (1) and a raw gas conduit (7) connected to an inlet of the compressor (1). Any gas compressor known to be suitable for compressing mixtures containing methane and carbon dioxide may be used, such as a turbo compressor, a piston compressor or preferably a screw compressor. The screw compressor may be a dry running compressor, or a fluid-cooled compressor cooled with water or oil. When an oil cooled compressor is used, the facility preferably also contains a droplet separator downstream of the compressor to prevent oil droplets from entering a membrane separation stage.

The facility of the invention comprises four membrane separation units (2) to (5). Each of the membrane separation units comprises a gas separation membrane having higher permeance for carbon dioxide than for methane, as well as a gas inlet, a retentate outlet and a permeate outlet. The term permeate here refers to a gas stream comprising the gas components of the gas stream fed to the membrane separation unit which have passed the gas separation membrane due to the difference in partial pressure across the membrane. The term retentate refers to the gas stream which remains after the gas components have passed the gas separation membrane. Since the gas separation membrane has higher permeance for carbon dioxide than for methane, the permeate will have a higher molar ratio of carbon dioxide to methane than the gas stream fed to the membrane separation unit, i.e. it will be enriched in carbon dioxide, and the retentate will have a higher molar ratio of methane to carbon dioxide than the gas stream fed to the membrane separation unit, i.e. it will be enriched in methane.

Suitable membranes which have higher permeability for carbon dioxide than for methane are known from the prior art. In general, membranes containing a separation layer of a glassy polymer, i.e. a polymer having a glass transition point at a temperature above the operating temperature of the membrane separation stage, will provide higher permeability for carbon dioxide than for methane. The glassy polymer may be a polyetherimide, a polycarbonate, a polyamide, a polybenzoxazole, a polybenzimidazole, a polysulfone or a polyimide and the gas separation membrane preferably comprises at least 80% by weight of a polyimide or a mixture of polyimides.

In a preferred embodiment, the gas separation membrane comprises at least 50% by weight of a polyimide prepared by reacting a dianhydride selected from 3,4,3′,4′-benzophenonetetracarboxylic dianhydride, 1,2,4,5-benzenetetracarboxylic dianhydride, 3,4,3′,4′-biphenyltetracarboxylic dianhydride, oxydiphthalic dianhydride, sulphonyldiphthalic dianhydride, 1,1,1,3,3,3-hexafluoro-2,2-propylidenediphthalic dianhydride and mixtures thereof with a diisocyanate selected from 2,4-tolylene diisocyanate, 2,6-tolylene diisocyanate, 4,4′-methylenediphenyl diisocyanate, 2,4,6-trimethyl-1,3-phenylene diisocyanate, 2,3,5,6-tetramethyl-1,4-phenylene diisocyanate and mixtures thereof. The dianhydride is preferably 3,4,3′,4′-benzophenonetetracarboxylic dianhydride or a mixture of 3,4,3′,4′-benzophenonetetracarboxylic dianhydride and 1,2,4,5-benzenetetracarboxylic dianhydride. The diisocyanate is preferably a mixture of 2,4-tolylene diisocyanate and 2,6-tolylene diisocyanate or a mixture of 2,4-tolylene diisocyanate, 2,6-tolylene diisocyanate and 4,4′-methylenediphenyl diisocyanate. Suitable polyimides of this type are commercially available from Evonik Fibres GmbH under the trade name P84® type 70, which has CAS number 9046-51-9 and is a polyimide prepared from 3,4,3′,4′-benzophenonetetracarboxylic dianhydride and a mixture of 64 mol % 2,4-tolylene diisocyanate, 16 mol % 2,6-tolylene diisocyanate and 20 mol % 4,4′-methylenediphenyl diisocyanate, and under the trade name P84® HT, which has CAS number 134119-41-8 and is a polyimide prepared from a mixture of 60 mol % 3,4,3′,4′-benzophenonetetracarboxylic dianhydride and 40 mol % 1,2,4,5-benzenetetracarboxylic dianhydride and a mixture of 80 mol % 2,4-tolylene diisocyanate and 20 mol % 2,6-tolylene diisocyanate. The gas separation membranes of this embodiment have preferably been heat treated in an inert atmosphere as described in WO 2014/202324 A1 to improve their long-term stability in the process of the invention.

In another preferred embodiment, the gas separation membrane comprises at least 50% by weight of a block copolyimide as described in WO 2015/091122 on page 6, line 20 to page 16, line 4. The block copolyimide preferably comprises at least 90% by weight of polyimide blocks having a block length of from 5 to 1000, preferably from 5 to 200.

The gas separation membrane may be flat membrane or a hollow fiber membrane and is preferably an asymmetrical hollow fiber membrane comprising a dense polyimide layer on a porous support. The term “dense layer” here refers to a layer which comprises essentially no macropores extending through the layer and the term “porous support” here refers to a support material having macropores extending through the support. The asymmetrical hollow fiber membrane can be prepared by coating a porous hollow fiber with a polyimide to form a dense polyimide layer on the support. In a preferred embodiment, the asymmetrical hollow fiber membrane is a membrane prepared in a phase inversion process by spinning with an annular two component spinning nozzle, passing a solution of a polyimide through the annular opening and a liquid containing a non-solvent for the polyimide through the central opening.

The gas separation membrane preferably comprises a dense separation layer of a glassy polymer coated with a dense layer of a rubbery polymer which rubbery polymer has higher gas permeability than the glassy polymer. The preferred gas separation membranes comprising a polyimide separation layer are preferably coated with a polydimethylsiloxane elastomer.

When the gas separation membrane is a flat membrane, the membrane separation units preferably comprise one or several spiral wound membrane modules containing the flat membranes and when the gas separation membrane is a hollow fiber membrane the membrane separation units preferably comprise one or several membrane modules containing a bundle of hollow fiber membranes. Each of the membrane separation units may comprise several membrane modules arranged in parallel and may also comprise several membrane modules arranged in series, wherein in a series of membrane modules the retentate provided by a membrane module is passed as feed to the membrane module subsequent in the series of membrane modules, the last membrane module of the series providing the retentate of the membrane separation stage, and the permeates of all membrane modules within a series are combined to provide the permeate of the membrane separation unit. When a membrane separation units comprises several membrane modules arranged in series, the membrane modules are preferably removable membrane cartridges arranged in series as a chain of cartridges in a common pressure vessel and connected to each other by a central permeate collecting tube, as described in detail in WO 2016/198450 A1. Membrane separation units which comprise several membrane modules arranged in parallel are preferred.

The facility of the invention comprises a feed conduit (8) connecting an outlet of the compressor (1) with the gas inlet of the first membrane separation unit (2). The feed conduit (8) preferably comprises a heat exchanger (19) arranged in the feed conduit for adjusting the temperature of the compressed gas to the operating temperature of the first membrane separation unit (2).

A dehumidifier may be arranged in the feed conduit. Such a dehumidifier is preferably configured to cool the compressed gas, condense water from the cooled gas in a condenser and reheat the gas. Reheating can be by compressed gas in a counter current heat exchanger.

The facility of the invention comprises a first retentate conduit (9) connecting the retentate outlet of the first membrane separation unit (2) to the gas inlet of the second membrane separation unit (3) and a second retentate conduit (10) connected to the retentate outlet of the second membrane separation unit (3). The second retentate conduit (10) preferably comprises a pressure regulating valve for adjusting or controlling the feed side pressure of the first membrane separation unit (2) and the second membrane separation unit (3).

A first permeate conduit (11) connects the permeate outlet of the first membrane separation unit (2) to the gas inlet of the third membrane separation unit (4). This first permeate conduit (11) preferably connects the permeate outlet of the first membrane separation unit (2) to the gas inlet of the third membrane separation unit (4) without any intermediary compressor or pump.

A third retentate conduit (12) connects the retentate outlet of the third membrane separation unit (4) to the gas inlet of the fourth membrane separation unit (5) and a fourth retentate conduit (13) connects the retentate outlet of the fourth membrane separation unit (5) to an inlet of the compressor (1). A pressure regulating valve (17) is preferably arranged in the fourth retentate conduit (13) for adjusting or controlling the feed side pressure of the third membrane separation unit (4) and the fourth membrane separation unit (5) as well as the permeate side pressure of the first membrane separation unit (2). If a multistage compressor is used, the fourth retentate conduit (13) may be connected to an inter-stage inlet of the compressor to reduce energy consumption for recompression.

A second permeate conduit (14) connects the permeate outlet of the second membrane separation unit (3) to an inlet of the compressor (1).

The facility of the invention comprises a third permeate conduit (15) connected to the permeate outlet of the third membrane separation unit (4). The third permeate conduit (15) is configured to discharge the third permeate to the surrounding atmosphere.

In a preferred embodiment the facility of the invention comprises means for direct or indirect measurement and/or means for controlling the methane concentration of the gas stream in the third permeate conduit (15), i.e. the third permeate stream. “Direct measurement” means an analytic method which analyses the gas composition of the third permeate stream. “Indirect measurement” means determining another process parameter, preferably of a gas stream, that can be correlated to the methane concentration in the third permeate stream. A preferred means for direct measurement is a methane concentration sensor (18) that is connected to the third permeate conduit (15) for monitoring the methane concentration in the third permeate stream. Any device known from the prior art to be suitable for determining the methane concentration in a gas mixture containing methane and carbon dioxide may be used as methane concentration sensor (18). Preferably, a commercial gas analyzer, measuring methane concentration by infrared absorption, or a process gas chromatograph are used as methane concentration sensor (18). Suitable means for indirect measurement are device to measure CO₂ and/or other components like O₂ and N₂ and assume the balance being methane. In addition, means being able to measure heating or caloric value of the gas. Examples are calorimeter like thermopile, micro combustion and residual oxygen combustion calorimeters.

The facility of the invention further comprises a methane oxidation unit (6) and a fourth permeate conduit (16) connecting the permeate outlet of the fourth membrane separation unit (5) to the methane oxidation unit (6). Any device known from the prior art to be suitable for oxidizing methane in a gas stream containing carbon dioxide as the major component may be used in the methane oxidation unit (6). The methane oxidation unit (6) preferably comprises a catalytic oxidizer, a regenerative thermal oxidizer or a biofilter.

The four membrane separation units (2) to (5) may contain the same membranes in all four membrane separation units or may contain different membranes in the membrane separation units. The membrane used in the first membrane separation unit (2) preferably has a pure gas selectivity of carbon dioxide over methane, determined at 20° C. and 5 bar, of at least 30, preferably from 40 to 120 and more preferably from 50 to 100. More preferably, all membrane separation units contain membranes having such high selectivity of carbon dioxide over methane. Suitable membrane modules and membrane cartridges containing hollow fiber polyimide membranes with such a high pure gas selectivity are commercially available from Evonik Fibres GmbH under the trade name SEPURAN® Green.

In a preferred embodiment, all membrane separation units contain the same membranes in the form of membrane modules of identical size arranged in parallel within a membrane separation unit. Different membrane areas are then provided in the membrane separation units by installing different numbers of membrane modules in a membrane separation unit. This embodiment has the advantage that only one membrane module type or, if modules with membrane cartridges are use, one membrane cartridge type must be kept in stock for replacing a defective membrane in the facility.

In another preferred embodiment, the fourth membrane separation unit (5) contains membranes having a higher permeance for carbon dioxide than the membranes used in the first membrane separation unit (2). In this embodiment, the membranes in the fourth membrane separation unit (5) may also have a lower pure gas selectivity for carbon dioxide over methane than the membranes used in the other membrane separation units. Using a more permeable membrane type with lower selectivity in the fourth membrane separation unit (5) can provide a desired methane content in the second permeate stream and a desired methane yield with considerably less membrane area and only a small increase of recycle rate compared to using the same membrane as in the first membrane separation unit (2). Membranes having a higher permeance for carbon dioxide and a lower selectivity may also be used in the second membrane separation unit (3) and/or the third membrane separation unit (4) if using less membrane area for separation has priority over providing low recycle rates for low operating costs. In a preferred embodiment the second membrane separation unit (3) contains membranes having a lower pure gas selectivity of carbon dioxide over methane compared to the first membrane separation unit (2) or compared to the first, third and fourth membrane separation units (2), (4) and (5).

Preferably, the membrane area of the second membrane separation unit (3) and of the fourth membrane separation unit (5) are selected to provide a separation capacity of the second membrane separation unit (3) which is larger than the separation capacity of the fourth membrane separation unit (5), the separation capacity of a membrane separation unit being the product of the membrane area of the membrane separation unit and the membrane permeance for carbon dioxide at 25° C. and a feed side pressure of 5 bar. Such a selection of membrane separation capacities provides a lower flow rate of the fourth permeate stream, which must be treated in the methane oxidation unit, when producing a third permeate stream of a target low methane concentration.

The second membrane separation unit (3) is preferably configured to provide counter-current flow on the permeate side relative to the feed side of the membrane. Preferably all membrane separation units of the facility of the invention are configured to provide such counter-current flow. Suitable membrane modules or cartridges with such counter-current flow are known from the prior art, for example from WO 2016/198450 or WO 2017/016913. Counter-current flow within a membrane module or cartridge provides better separation with a higher purity of the retentate produced by the membrane separation unit.

The facility of the invention is configured to provide a carbon dioxide concentration in the gas stream in first permeate conduit (11), i.e. the first permeate stream, in a range of from 90 to 99% by volume. Preferably the facility comprises means for controlling the permeate side pressure in the first membrane separation unit (2) and/or the separation capacities in the four membrane separation units (2) to (5) to provide a carbon dioxide concentration in the first permeate stream of from 90 to 99% by volume. Even more preferred the permeate side pressure in the first membrane separation unit (2) and the separation capacities, which are the product of the membrane area and the membrane permeance for carbon dioxide at a temperature of 25° C. and a feed side pressure of bar, in the four membrane separation units (2) to (5) are configured to provide a carbon dioxide concentration in the first permeate stream of from 90 to 99% by volume.

In a preferred embodiment, the facility of the invention further comprises a controller connected to the methane concentration sensor (18) which controls at least one process parameter for maintaining the concentration of methane in the third permeate stream at or below a target value. Adjusting the operating conditions of the facility based on measuring the methane concentration in the third permeate stream allows for adhering to a limit for methane emission even when the composition or the flow rate of the raw gas stream changes.

In a first alternative, the process parameter is the permeate side pressure of the first membrane separation unit (2). The facility of the invention then comprises a pressure regulating valve (17) arranged in the fourth retentate conduit (13) and the controller controls the pressure regulating valve (17) based on data measured by the methane concentration sensor (18). The controller controls the pressure regulating valve (17) to decrease the permeate side pressure of the first membrane separation unit (2) when the concentration of methane in the third permeate stream rises to above the target value This embodiment has the advantage of requiring little extra equipment. Placing the pressure regulating valve (17) in the fourth retentate conduit (13) is advantageous compared to placing the pressure regulating valve (17) in the third retentate conduit (12) or in the first permeate conduit (11), because it requires less membrane area in the third membrane separation unit (4) and the fourth membrane separation unit (5) than for the alternatives for placing the pressure regulating valve.

In a second alternative, the process parameter is the feed stream temperature. The facility of the invention then comprises a heat exchanger (19) in the feed conduit (8) and a flow regulating valve (20) controlling flow of a heating or cooling fluid to the heat exchanger (19) and the controller controls this flow regulating valve (20) based on data measured by the methane concentration sensor (18). The controller controls the heat exchanger (19), preferably via regulating valve (20) to decrease the temperature of the feed stream when the concentration of methane in the third permeate stream rises to above the target value. This embodiment is advantageous for operating the facility at reduced load, because recycle rates will be lower at reduced load compared to a facility where the permeate pressure of the first membrane separation unit (2) is adjusted at reduced load. The flow regulating valve (20) may be placed in a conduit passing the heating or cooling fluid to the heat exchanger (19). When the facility comprises a dehumidifier in the feed conduit (8), the heat exchanger (19) may be a part of the dehumidifier or may be present in addition to the dehumidifier. In a preferred embodiment, the second retentate conduit (10) is connected to a cooling fluid inlet of the heat exchanger (19) and the flow regulating valve is placed in a bypass conduit connected to the second retentate conduit (10). This allows for cooling the feed stream with the second retentate stream, controlling the temperature of the feed stream by controlling the fraction of the second retentate stream which passes through heat exchanger (19). This alternative has the advantage that no additional energy is needed for cooling the feed stream.

In a third alternative, the process parameter is the membrane area in use in the third membrane separation unit (4). The facility of the invention then comprises a multitude of membrane modules arranged in parallel in the third membrane separation unit (4) with at least one of these membrane modules comprising shut-off valves which block flow through the membrane module. The controller then controls the shut-off valves based on data measured by the methane concentration sensor (18) to close shut-off valves of membrane module(s) when the concentration of methane in the third permeate stream rises to above the target value. Flow through a membrane module can be blocked by shut-off valves on at least two of the gas inlet, the retentate outlet and the permeate outlet of the membrane module, with shut-off valves on the gas inlet and the permeate outlet being preferred. Slowly closing shut-off valves are preferred to prevent a pressure surges which can cause membrane damage. This embodiment is advantageous where the flow rate or the in composition of the gas stream shows large variation over time, as is typically the case for a landfill gas or a fermentation which uses varying feedstocks.

In a fourth alternative, the process parameter is the operation mode of a module in the first membrane separation unit (2). The facility of the invention then comprises a bore-side fed hollow fiber membrane module in the first membrane separation unit (2) with the gas inlet on a first end of the module, the retentate outlet on a second end of the module opposite to the first end, the first permeate outlet adjacent to the first end of the module and connected to the first permeate conduit (11) and an additional permeate outlet adjacent to the second end of the module. The facility then further comprises an additional conduit (21) which connects the additional permeate outlet with the gas inlet of the fourth membrane separation unit (5) and a flow regulating valve (22) arranged in the additional conduit (21) and the controller controls this flow regulating valve (22) based on data measured by the methane concentration sensor (18) to decrease the flow through the additional conduit (21) when the concentration of methane in the third permeate stream rises to above the target value.

The process of the invention is carried out in a facility of the invention as described above.

A raw gas stream, which contains from 20 to 60% by volume, preferably 20 to 50%, by volume carbon dioxide and has a combined content of methane and carbon dioxide of at least 95% by volume, is introduced into the raw gas conduit (7) of the facility. The raw gas may be a natural gas or a landfill gas or preferably a biogas from an anaerobic digester. The raw gas preferably comprises from 30 to 50% by volume carbon dioxide. The raw gas is preferably a desulfurized biogas from an anaerobic digester. Desulfurizing the raw gas stream prevents corrosion of the compressor and of gas conduits of the facility. The biogas may also be pretreated by drying and/or by adsorption of volatile organic compounds, such as volatile siloxanes, on an adsorbent. When the raw gas is a biogas from an anaerobic digester operated with controlled air addition to reduce hydrogen sulfide formation in the digester, the raw gas will typically contain minor amounts of oxygen and nitrogen.

The raw gas stream is combined with recycle streams from the fourth retentate conduit (13) and the second permeate conduit (14) and is compressed with compressor (1) to provide a feed stream at a feed pressure of from 7 to 25 bar and a temperature of from 15 to 50° C. Compressing will typically increase the temperature of the gas to a value higher than desired for operating the first membrane separation unit (2) and therefore the compressed gas will typically be cooled to provide the feed stream at the required temperature. The compressed gas may also be dehumidified by cooling it to a temperature lower than desired for operating the first membrane separation unit (2), condensing water from the compressed gas at this low temperature and reheating the gas after separation of the condensed water to the required temperature. The compressed gas is preferably dehumidified with a dehumidifier arranged in the feed conduit as described above. Dehumidifying the compressed gas prevents condensation of water in a membrane separation unit which would reduce the separation capacity of the membrane separation unit.

The feed stream is then separated in the first membrane separation unit (2) into a first permeate stream and a first retentate stream, using a membrane which has a mixed gas selectivity for carbon dioxide over methane of at least 30 and preferably of from 40 to 100, more preferably of from 40 to 80, at the feed pressure and the temperature of the feed stream. Suitable membrane modules and membrane cartridges containing hollow fiber polyimide membranes with such a high mixed gas selectivity are commercially available from Evonik Fibres GmbH under the trade name SEPURAN® Green. The permeate side pressure in the first membrane separation unit and the separation capacities in the four membrane separation units are selected to provide a carbon dioxide concentration in the first permeate stream of from 90 to 99% by volume. The separation capacity of a membrane separation unit is the product of the membrane area and the membrane permeance for carbon dioxide at a temperature of 25° C. and a feed side pressure of 5 bar, as defined further above. The selection of suitable values for the permeate side pressure in the first membrane separation unit and the separation capacities in the four membrane separation units can be carried out with process simulation software which calculates mass transfer of the gas components through the membrane by numerical integration of the known differential equations for mass transfer through a membrane by a solution-diffusion process based on experimental data for the permeance of the membrane for methane and carbon dioxide. Such calculations are preferably carried out with boundary conditions set for the target values for the methane concentration in the third permeate stream, the carbon dioxide concentration in the second retentate stream and the methane recovery with the second retentate stream. The temperature dependency of permeation can be accounted for by applying the equations known from M. Scholz et. al, Ind. Eng. Chem. Res. 52 (2013) 1079-1088.

The first retentate stream is separated in the second membrane separation unit (3) into a second retentate stream and a second permeate stream. The second retentate stream is further processed or withdrawn as a methane rich product stream, preferably withdrawn as a methane rich product stream. A non limiting list of examples for further processing comprises odorization, heat value adjustment, pressure adjustment, processing to compressed natural gas or liquified natural gas, grid injection, polishing (removing <0.5% components down to ppm levels), electricity generation, or at least use a split stream and process according to one of the a fore mentioned options. The second retentate stream is preferably withdrawn or forwarde3d to further processing through a second retentate conduit (10) which comprises a pressure regulating valve in the conduit and a constant retentate pressure is maintained with this valve. The second permeate stream is recycled through the second permeate conduit (14). An additional pressure regulating valve may be placed in the second permeate conduit (14) to adjust or control the permeate pressure of the second membrane separation unit (3). The separation capacity of the second membrane separation unit (3) is preferably selected to provide a carbon dioxide concentration in the second retentate stream of from 0.5 to 4.0% by volume. It is also preferred to select the separation capacity of the second membrane separation unit (3) to provide a carbon dioxide concentration in the second permeate stream of from 81 to 89% by volume carbon dioxide. Such selection can be made by a process simulation as described above, using target values within these ranges for the carbon dioxide concentration in the second retentate stream and/or the second permeate stream as boundary conditions for the process simulation.

The first permeate stream is separated in the third membrane separation unit (4) into a third retentate stream and a third permeate stream and the third permeate stream is discharged to the surrounding atmosphere without further methane removal. The separation capacity of the third membrane separation unit (4) is preferably selected to provide a carbon dioxide concentration in the third permeate stream of 0.3% by volume or less, preferably from 0.1 to 0.2% by volume. Such a selection can be made by a process simulation as described above, using a target value within this range for the carbon dioxide concentration in the third permeate stream as a boundary condition for the process simulation. The third permeate stream is preferably discharged through a third permeate conduit (15) with a methane concentration sensor (18) connected to the third permeate conduit (15) and the carbon dioxide concentration in the third permeate stream is monitored.

The third retentate stream is separated in the fourth membrane separation unit (5) into a fourth retentate stream and a fourth permeate stream and the fourth retentate stream is recycled through the retentate conduit (13). The separation capacity of the fourth membrane separation unit (5) is preferably selected to provide a methane recovery with the second retentate stream of from 98.0 to 99.9%, preferably in combination with a carbon dioxide concentration in the second retentate stream of from 0.5 to 4.0% by volume. Such a selection can be made by a process simulation as described above, using a target value for the methane recovery within this range as a boundary condition for the process simulation. Preferably, the separation capacities of the second membrane separation unit (3) and the fourth membrane separation unit (5) are selected to provide a separation capacity of the second membrane separation unit (3) which is from 1.2 to 8 times the separation capacity of the fourth membrane separation unit (5). Such a selection of membrane separation capacities provides a lower flow rate of the fourth permeate stream, which must be treated in the methane oxidation unit, when producing a third permeate stream of a target low methane concentration.

The fourth permeate stream is passed to the methane oxidation unit (6) and is oxidized in this unit to provide an off-gas stream containing less than 0.3% by volume methane, which off-gas stream is discharged to the surrounding atmosphere. Methane is preferably oxidized in the methane oxidation unit (6) with an oxygen containing gas as the oxidant, preferably with air. The oxygen containing gas can be mixed with the fourth permeate stream before introducing it to the methane oxidation unit (6) or can be supplied separately to the methane oxidation unit (6). Methane is preferably oxidized with a catalytic oxidizer, a regenerative thermal oxidizer or a biofilter. In a preferred embodiment, the methane oxidation unit (6) comprises a catalytic oxidizer or a regenerative thermal oxidizer and the separation capacity of the fourth membrane separation unit is selected to provide a methane concentration in the fourth permeate stream which allows autothermal operation of the oxidizer.

The process of the invention allows for adhering to strict limits for methane emission to the atmosphere with only a small methane oxidation unit, because the flow rate of the fourth permeate stream treated in the methane oxidation unit is typically lower than the flow rate of the third permeate stream which can be discharged without treatment. The process can provide high methane yields based on the raw gas even for operating the methane oxidation unit as an autothermal catalytic oxidizer or a regenerative thermal oxidizer without supply of additional fuel.

Using a membrane with a mixed gas selectivity of at least 30 in the first membrane separation unit (2) and adjusting separation capacities to provide a carbon dioxide concentration in the first permeate stream of from 90 to 99% by volume allows for separating a larger proportion of the carbon dioxide contained in the raw gas stream with the third permeate stream at a low methane concentration of 0.3% by volume and thereby reduces the flow rate of the fourth permeate stream and as a consequence the size of the methane oxidation unit (6).

Selecting the separation capacity of the second membrane separation unit (3) to provide a carbon dioxide concentration of from 0.5 to 4.0% by volume in the second retentate stream and of from 81 to 89% by volume in the second permeate stream increases the fraction of carbon dioxide removed with the third permeate stream and reduces the overall recycle rate in the process.

In a preferred embodiment of the process of the invention, the feed pressure and the permeate side pressure of the first membrane separation unit (2) are selected to provide a pressure ratio in the third membrane separation unit (4) which is from 0.4 to 1.2 times and preferably from 0.4 to 1.0 times the pressure ratio in the first membrane separation unit (2). The pressure ratio in a membrane unit is defined here as the ratio between the feed side pressure and the permeate side pressure in the membrane unit. Such a selection of pressure ratios allows for operating the process with a lower overall recycle rate.

In another preferred embodiment of the process of the invention, the concentration of methane in the third permeate stream is measured with a methane concentration sensor (18) and an operating parameter of the separation process is adjusted based on the measured value to maintain the concentration of methane in the third permeate stream at or below a target value, preferably a target value in the range of from 0.1 to 0.3% by volume. Preferably, an operating parameter of the first membrane separation unit (2) is adjusted. This allows for maintaining the methane concentration in the third permeate stream below a regulatory limit for methane emission even when the composition of the raw gas stream or the flow rate of the raw gas stream changes.

Preferably, the permeate side pressure of the first membrane separation unit (2) is adjusted based on the measured concentration of methane in the third permeate stream, decreasing the permeate side pressure when the concentration of methane in the third permeate stream rises to above the target value. This will typically be the case when the flow rate of the raw gas stream decreases or the methane content of the raw gas stream increases (see Example 10 in comparison with Example 6). The permeate side pressure of the first membrane separation unit (2) is preferably controlled with a pressure regulating valve (17) arranged in the fourth retentate conduit (13). The permeate side pressure is preferably controlled to maintain the concentration of methane in the third permeate stream essentially constant with a variation of the methane concentration of no more than 0.03% by volume.

In another preferred embodiment, the temperature of the feed stream is adjusted based on the measured concentration of methane in the third permeate stream, decreasing the temperature of the feed stream when the concentration of methane in the third permeate stream rises to above the target value. The temperature of the feed stream can be adjusted by adjusting the cooling of the gas stream leaving the compressor. When the compressed gas is dehumidified by cooling and condensing water as described further above, the temperature of the feed stream can also be adjusted by adjusting the reheating of the compressed gas after the condensation step. Alternatively, the temperature of the first permeate stream is adjusted based on the measured concentration of methane in the third permeate stream, decreasing the temperature of the first permeate stream when the concentration of methane in the third permeate stream rises to above the target value. Both these alternatives have the advantage that operating the process at a reduced flow rate of the raw gas stream will lead to less increase in the recycle rate compared to the alternative of adjusting the permeate side pressure of the first membrane separation unit (2). For both alternatives the temperature is preferably controlled to maintain the concentration of methane in the third permeate stream essentially constant with a variation the methane concentration of no more than 0.03% by volume. In both alternatives the temperature can be decreased by heat exchange with the second retentate stream and the temperature can be adjusted by controlling the fraction of the second retentate stream used for this heat exchange. Using the second retentate stream for cooling the feed stream or the first permeate stream has the advantage that no extra energy is needed for adjusting the temperature.

In yet another preferred embodiment, the process is carried out in a facility which comprises a multitude of membrane modules arranged in parallel in the third membrane separation unit (4) with at least one of these membrane modules comprising shut-off valves which block flow through the membrane module and shut-off valves of a membrane module are closed when the measured concentration of methane in the third permeate stream rises to above a target value.

In still another preferred embodiment, the process is carried out in a facility where the first membrane separation unit (2) comprises a bore-side fed hollow fiber membrane module with the first permeate outlet adjacent to one end of the module and an additional permeate outlet, adjacent to the opposite end of the module, connected to the gas inlet of the fourth membrane separation unit (5) by an additional conduit (21), as described further above. The flow through the additional conduit (21) is then controlled with a flow regulating valve (22) arranged in the additional conduit (21) based on the measured concentration of methane in the third permeate stream, decreasing flow through the additional conduit (21) when the concentration of methane in the third permeate stream rises to above the target value.

These different alternatives for adjusting an operating parameter of the separation process based on the measured concentration of methane in the third permeate stream may also be combined with each other to maintain an essentially constant concentration of methane in the third permeate stream over a broader range of raw gas compositions and flow rates of the raw gas stream. Preferred are combinations where the alternative of blocking flow through one or several membrane modules arranged in parallel in the third membrane separation unit (4), which allows adjusting over a large range but only in discrete steps, is combined with adjusting the permeate side pressure, the temperature of the feed stream or the temperature of the first permeate stream, in particular adjusting these operating parameters in narrow ranges bridging only the gaps between operating the third membrane separation unit (4) with a different number of membrane modules in use.

The following examples demonstrate the invention and its advantages.

### EXAMPLES

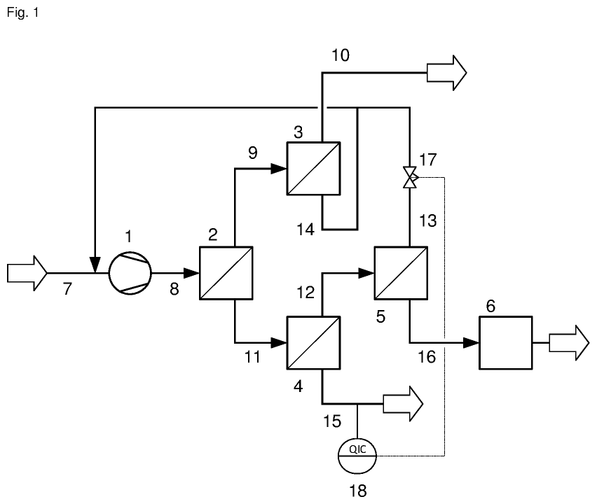

Calculations were carried out for gas separation in a facility as shown in FIG. 1, using process simulation software which calculates mass transfer of the gas components through the membrane by numerical integration of the known differential equations for mass transfer through a membrane by a solution-diffusion process, based on experimental data for the permeance of the membrane for methane and carbon dioxide. All pressures are given as absolute pressure.

The simulation underlying the examples were conducted under the premise that methane concentration in the 3rd permeate stream is set, measured and controlled to be at 0.2 vol. % respectively 0.3 vol. %. The specific value is given in the examples.

### Comparative Example 1

WO 2015/036709 A1 provides a facility and method, which can be used to purify biogas. According to page 1, paragraph 6 of WO′709 biogas typically comprise 30 to 75% methane, 15 to 60% CO₂, 0 to 15% N₂ and 0 to 5% O₂. WO'709 further discloses on page 3, last paragraph that the method should enable the production of a gas containing more than 85%, preferably more than 95% and more preferred more than 97.5% methane. WO′709, page 7, provides a table, which shows methane yields and recycling rates for a two, a three, a four and a five-units membrane separation process. WO '709, however, does not disclose
how these yields and recycling rates were achieved,
which raw gas mixture was used,
which membranes were used,
which process pressures and temperatures were used.

Since WO '709 does not comprise examples that could be reproduced to compare the method and facility with the present invention, Comparative Examples 1a and 1b were based on the rudimentary information summarized above. Process simulations were carried out in Comparative Examples 1a and 1 b with the goal to match a CH₄ rendement of 99.09% and a recycling rate of 1.42, as given for the four-units process in the Table on page 7 of WO '709. Since it is unclear what “rendement” exactly means, it could mean “content” or it could mean a “yield”, Comparative Example 1a was prepared with a CH₄ content of 99.09% in the methane enriched product stream as boundary condition and Comparative Example 1b has a CH₄ yield in the methane-rich stream of 99.09% as boundary condition.

### Comparative Example 1a

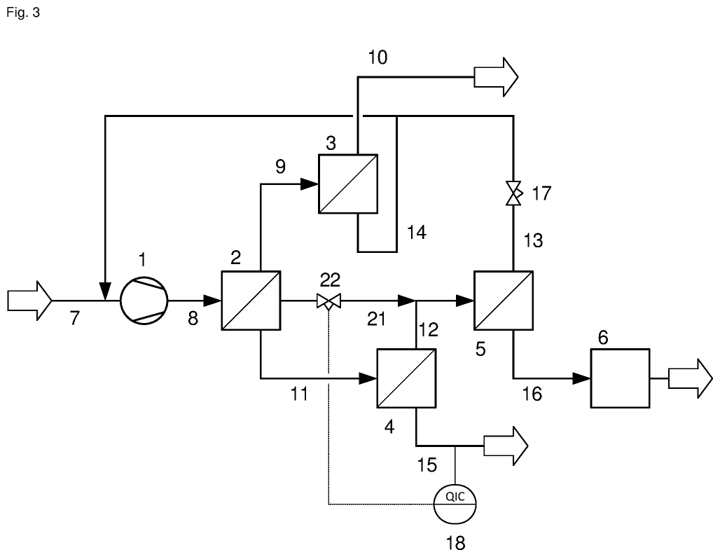

A raw gas stream was provided at 1.01 bar pressure with a flow rate of 5,420 Nm³/h and contained 50% by volume of methane, 49.7% by volume of carbon dioxide, 0.2% by volume of nitrogen and 0.1% by volume of oxygen. The raw gas stream was subjected to membrane separation process in a facility according to FIG. 3 of WO '709, containing 367 SEPURAN® Green membrane modules, each module containing membranes with a mixed gas selectivity for carbon dioxide over methane of 50, for carbon dioxide over oxygen of 5.0 and for carbon dioxide over nitrogen of 31 and having a separation capacity of 2.101 mol s⁻¹ MPa⁻¹. Feed temperature was set to 25° C. and feed pressure to 16 bar. Calculations were carried out for isothermal separation assuming a pressure drop of 70 mbar on the retentate side of a module. The simulation was carried out with the boundary conditions of providing a methane content of 99.09% by volume in the second retentate stream and a recycling rate of 42% in sum for all recycled gas streams. 137 membrane modules in the first membrane separation unit, 83 membrane modules in the second membrane separation unit, 62 membrane modules in the third membrane separation unit and 85 membrane modules in the fourth membrane separation unit were used. The calculated flow rates and compositions of the process streams are given in Table 1.

TABLE 1

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 5420 | 1.01 | 25.0 | 49.70 | 50.00 | 0.20 | 0.10 |
| Feed | 7713 | 16.04 | 22.8 | 53.92 | 45.66 | 0.21 | 0.21 |
| First | 3261 | 16.02 | 13.5 | 6.38 | 93.05 | 0.39 | 0.18 |
| retentate | | | | | | | |
| First | 4452 | 2.73 | 17.6 | 88.75 | 10.95 | 0.07 | 0.23 |
| permeate | | | | | | | |
| Second | 2715 | 16.00 | 11.7 | 0.43 | 99.09 | 0.39 | 0.09 |
| retentate | | | | | | | |
| Second | 546 | 1.01 | 12.7 | 35.94 | 63.00 | 0.41 | 0.65 |
| permeate | | | | | | | |
| Third | 3078 | 2.44 | 17.1 | 83.98 | 15.62 | 0.11 | 0.29 |
| retentate | | | | | | | |
| Third | 1374 | 1.01 | 17.4 | 99.42 | 0.48 | 0.01 | 0.09 |
| permeate | | | | | | | |
| Fourth | 1747 | 2.30 | 16.2 | 72.62 | 26.79 | 0.18 | 0.41 |
| retentate | | | | | | | |
| Fourth | 1331 | 1.01 | 16.8 | 98.89 | 0.96 | 0.01 | 0.14 |
| permeate | | | | | | | |

The raw gas stream used in Comparative Example 1a meets the “biogas specification” of WO'709 and the methane content in the second retentate stream is above 97.5% as required in WO'709, too. Both, recycling rate of 1.42 (7713 Nm³/h (feed stream)/5420 Nm³/h (raw gas stream)=1.42) and methane content in the second retentate stream of 99.09%, correspond to the discloser in the Table on page 7 of WO'709, if “rendement” means yield.

Table 1 shows that the CO₂ content of the 1^{st} permeate stream is 88.75% and thus, outside the range claimed in of the present invention. The methane content in the 3rd permeate stream is 0.48%. As consequence, the process of WO'709 cannot be used in locations with strong regulators requirements on methane emission, i.e. the methane content in the off-gas streams, without subjecting both the 3rd and the 4^{th} permeate stream to a methane reducing post treatment step.

### Comparative Example 1b

Comparative Example 1a was reproduced with identical raw gas stream, type of membranes, feed temperature and feed pressure. Calculations were carried out for isothermal separation assuming a pressure drop of 70 mbar on the retentate side of a module. The simulation was carried out with the boundary conditions of providing a methane yield of 99.09% and a recycling rate of 42% in sum for all recycled gas streams. 137 membrane modules in the first membrane separation unit, 83 membrane modules in the second membrane separation unit, 62 membrane modules in the third membrane separation unit and 85 membrane modules in the fourth membrane separation unit were used. The calculated flow rates and compositions of the process streams are given in Table 2.

TABLE 2

| | Flow | | | Concentration | | | |
|---|---|---|---|---|---|---|---|
| | rate | Pressure | Temperature | [% by volume] | | | |
| Gas stream | [Nm³/h] | [bar] | [° C.] | Methane | CO₂ | Nitrogen | Oxygen |
| Raw gas | 4870 | 1.01 | 25 | 50 | 49.7 | 0.2 | 0.1 |
| Feed | 6900 | 16.04 | 22.8 | 47.21 | 52.36 | 0.22 | 0.21 |
| First | 2920 | 16.02 | 13.5 | 94.48 | 4.95 | 0.40 | 0.17 |
| retentate | | | | | | | |
| First | 3980 | 2.65 | 17.7 | 12.52 | 87.15 | 0.09 | 0.24 |
| permeate | | | | | | | |
| Second | 2430 | 16 | 11.8 | 99.29 | 0.25 | 0.39 | 0.07 |
| retentate | | | | | | | |
| Second | 490 | 1.01 | 12.8 | 70.63 | 28.29 | 0.45 | 0.63 |
| permeate | | | | | | | |
| Third | 2717 | 2.4 | 17.1 | 18.07 | 81.50 | 0.12 | 0.31 |
| retentate | | | | | | | |
| Third | 1263 | 1.01 | 17.4 | 0.59 | 99.30 | 0.01 | 0.10 |
| permeate | | | | | | | |
| Fourth | 1540 | 2.27 | 16.3 | 30.93 | 68.44 | 0.20 | 0.43 |
| retentate | | | | | | | |
| Fourth | 1177 | 1.01 | 16.9 | 1.24 | 98.59 | 0.01 | 0.16 |
| permeate | | | | | | | |
| Methane | 99.09% | | | | | | |
| yield | | | | | | | |

The raw gas stream used in Comparative Example 1a meets the “biogas specification” of WO'709 and the methane content in the second retentate stream is above 97.5% as required in WO'709, too. Both, recycling rate of 1.42 (6900 Nm³/h (feed stream)/4870 Nm³/h (raw gas stream)=1.42) and methane yield in the second retentate stream of 99.09%, correspond to the discloser in the Table on page 7 of WO'709, if “rendement” means yield.

Table 2 shows that the CO₂ content of the 1^{st} permeate stream is 87.15%, and thus, outside the range claimed in of the present invention. The methane content in the 3rd permeate stream is 0.59. As consequence, the process of WO'709 cannot be used in locations with strong regulators requirements on methane emission, i.e. the methane content in the off-gas streams, without subjecting both the 3^{rd} and the 4^{th} permeate stream to a methane reducing post treatment step.

### Example 1

Gas separation was calculated for separating a raw gas stream provided at 1.01 bar with a flow rate of 10,000 Nm³/h and containing 49.9% by volume of methane, 50% by volume of carbon dioxide and 0.1% by volume of oxygen in a facility containing 330 SEPURAN® Green membrane modules, each module containing membranes with a mixed gas selectivity for carbon dioxide over methane of 50, for carbon dioxide over oxygen of 5.0 and for carbon dioxide over nitrogen of 31 and having a separation capacity of 2.101 mol s⁻¹ MPa⁻¹. Feed temperature was set to 25° C. and feed pressure was set to 16 bar. Calculations were carried out for isothermal separation assuming a pressure drop of 70 mbar on the retentate side of a module. An optimization was carried out with the boundary conditions of providing a methane content of 97.0% by volume in the second retentate stream, a methane content of 0.2% by volume in the third permeate stream, a methane yield with the second retentate stream of 99.8% and a flow rate of the fourth permeate stream of 550 Nm³/h. Permeate side pressure of the first membrane separation unit and distribution of membrane modules to the four membrane separation units were varied to provide a minimum recycle rate (combined second permeate stream and fourth retentate stream relative to raw gas stream). The optimization calculated a minimum for the recycle rate at 46.0% for a permeate side pressure of the first membrane separation unit of 3.48 bar and a distribution of 59.8 membrane modules in the first membrane separation unit, 126.6 membrane modules in the second membrane separation unit, 118.1 membrane modules in the third membrane separation unit and 25.4 membrane modules in the fourth membrane separation unit. The calculated flow rates and compositions of the process streams are given in table 3.

The calculation shows that the process of the invention can upgrade a typical biogas to biomethane having a methane content of 97% by volume with a methane yield of 99.8% with a recycle rate of only 46%. The process of the invention separates the major part of the carbon dioxide with a gas stream containing only 0.2% by volume of methane which can be discharged directly to the atmosphere. Only a small off-gas stream with a flow rate of 6% relative to the biogas must be treated in a methane oxidation unit. This methane oxidation unit can be operated as an autothermal catalytic oxidizer or a regenerative thermal oxidizer without an additional fuel supply because the off-gas stream contains 1.7% by volume of methane.

TABLE 3

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 50.0 | 49.9 | 0.1 |
| Feed | 14599 | 60.95 | 38.91 | 0.15 |
| First | 9491 | 41.53 | 58.30 | 0.18 |
| retentate | | | | |
| First | 5106 | 96.94 | 2.97 | 0.09 |
| permeate | | | | |
| Second | 5134 | 2.86 | 97.00 | 0.18 |
| retentate | | | | |
| Second | 4353 | 86.96 | 12.81 | 0.23 |
| permeate | | | | |
| Third | 795 | 81.64 | 18.03 | 0.34 |
| retentate | | | | |
| Third | 4311 | 99.76 | 0.20 | 0.04 |
| permeate | | | | |
| Fourth | 245 | 44.50 | 54.86 | 0.64 |
| retentate | | | | |
| Fourth | 550 | 98.11 | 1.68 | 0.20 |
| permeate | | | | |

### Comparative Example 2

The calculation of Example 1 was repeated with the following modifications: Membranes having a mixed gas selectivity for carbon dioxide over methane of 20, for carbon dioxide over oxygen of 5 and for carbon dioxide over nitrogen of 56 and having a separation capacity of 2.101 mol s⁻¹ MPa⁻¹ were used in the first separation unit (2) and 108 instead of 118 modules were used in the third separation unit (4).

Gas separation was calculated for separating a raw gas stream provided at 1.01 bar with a flow rate of 10,000 Nm³/h and containing 49.9% by volume of methane, 50% by volume of carbon dioxide and 0.1% by volume of oxygen. SEPURAN® Green membrane modules, each module containing membranes with a mixed gas selectivity for carbon dioxide over methane of 50, for carbon dioxide over oxygen of 5.0 and for carbon dioxide over nitrogen of 31 and having a separation capacity of 2.101 mol s⁻¹ MPa⁻¹ were used in the second, third and fourth separation units (3), (4) and (5). Feed temperature was set to 25° C. and feed pressure was set to 16 bar. Calculations were carried out for isothermal separation assuming a pressure drop of 70 mbar on the retentate side of a module. 60 membrane modules in the first membrane separation unit, 127 membrane modules in the second membrane separation unit, 108 membrane modules in the third membrane separation unit and 25 membrane modules in the fourth membrane separation unit. The calculated flow rates and compositions of the process streams are given in Table 4.

TABLE 4

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 50.0 | 49.9 | 0.1 |
| Feed | 18559 | 68.56 | 31.30 | 0.14 |
| First | 11640 | 52.54 | 47.28 | 0.18 |
| retentate | | | | |
| First | 6920 | 95.51 | 4.42 | 0.07 |
| permeate | | | | |
| Second | 5221 | 4.48 | 95.35 | 0.17 |
| retentate | | | | |
| Second | 6418 | 91.63 | 8.19 | 0.18 |
| permeate | | | | |
| Third | 2979 | 89.89 | 9.99 | 0.12 |
| retentate | | | | |
| Third | 3940 | 99.77 | 0.21 | 0.02 |
| permeate | | | | |
| Fourth | 2141 | 86.10 | 13.75 | 0.15 |
| retentate | | | | |
| Fourth | 838 | 99.55 | 0.40 | 0.04 |
| permeate | | | | |

Table 4 shows that a methane content in the 3^{rd} permeate stream of 0.21% can be obtained by use of lower selective membranes in the 1^{st} separation unit, too, but the process becomes much less efficient. The recycling rate of 85.6% in Comparative Example 2 is nearly twice as high than the 46% of Example 1 and the methane content in the 2^{nd} retentate stream is decreased to 95.35%.

### Example 2

The calculation of Example 1 was repeated with the following modifications:

In the second membrane separation unit (3) membranes having a mixed gas selectivity for carbon dioxide over methane of 20, for carbon dioxide over oxygen of 15 and for carbon dioxide over nitrogen of 169 and having a separation capacity of 6.303 mol s⁻¹ MPa⁻¹ were used. 42 instead of 127 modules were used in the second membrane separation unit (3).

As in Example 1, gas separation was calculated for separating a raw gas stream provided at 1.01 bar with a flow rate of 10,000 Nm³/h and containing 49.9% by volume of methane, 50% by volume of carbon dioxide and 0.1% by volume of oxygen. SEPURAN® Green membrane modules, each module containing membranes with a mixed gas selectivity for carbon dioxide over methane of 50, for carbon dioxide over oxygen of 5.0 and for carbon dioxide over nitrogen of 31 and having a separation capacity of 2.101 mol s⁻¹ MPa⁻¹ were used in the first, the third and the fourth separation units (2), (4) and (5). Feed temperature was set to 25° C. and feed pressure was set to 16 bar. Calculations were carried out for isothermal separation assuming a pressure drop of 70 mbar on the retentate side of a module. 60 membrane modules in the first membrane separation unit, 42 membrane modules in the second membrane separation unit, 108 membrane modules in the third membrane separation unit and 25 membrane modules in the fourth membrane separation unit. The calculated flow rates and compositions of the process streams are given in Table 5.

TABLE 5

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 50.0 | 49.9 | 0.1 |
| Feed | 16241 | 59.92 | 39.99 | 0.09 |
| First | 11095 | 42.75 | 57.15 | 0.10 |
| retentate | | | | |
| First | 5146 | 96.95 | 3.00 | 0.05 |
| permeate | | | | |
| Second | 5125 | 2.81 | 97.03 | 0.16 |
| retentate | | | | |
| Second | 5970 | 77.03 | 22.92 | 0.05 |
| permeate | | | | |
| Third | 845 | 82.57 | 17.24 | 0.19 |
| retentate | | | | |
| Third | 4301 | 99.77 | 0.20 | 0.03 |
| permeate | | | | |
| Fourth | 271 | 49.06 | 50.59 | 0.35 |
| retentate | | | | |
| Fourth | 574 | 98.37 | 1.52 | 0.11 |
| permeate | | | | |

Table 5 shows that if lower selective membranes are used in the second separation unit (3), in contrast to using such membranes in the first separation unit (2) as in Comparative Example 2, significant increase of the volume flow of the fourth permeate stream compared to Example 1 can be avoided. Also, the methane target contents of 97% in the second retentate and of 0.21% in the third permeate stream can be reached analogue to Example 1.

### Example 3

The calculation of example 1 was repeated changing the boundary condition for the flow rate of the fourth permeate stream to 1000 Nm³/h. The optimization calculated a minimum for the recycle rate at 39.1% for a permeate side pressure of the first membrane separation unit of 3.51 bar and a distribution of 69.6 membrane modules in the first membrane separation unit, 118.2 membrane modules in the second membrane separation unit, 104.5 membrane modules in the third membrane separation unit and 34.5 membrane modules in the fourth membrane separation unit. The calculated flow rates and compositions of the process streams are given in table 6.

The calculation shows that there is a trade-off between providing a low recycle rate and reducing the size of the off-gas stream which must be treated in the methane oxidation unit.

TABLE 6

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 50.0 | 49.9 | 0.1 |
| Feed | 13914 | 58.94 | 40.91 | 0.15 |
| First | 8660 | 36.21 | 63.60 | 0.18 |
| retentate | | | | |
| First | 5106 | 96.94 | 2.97 | 0.09 |
| permeate | | | | |
| Second | 5134 | 2.87 | 97.00 | 0.13 |
| retentate | | | | |
| Second | 3522 | 84.65 | 15.09 | 0.25 |
| permeate | | | | |
| Third | 1391 | 86.64 | 13.10 | 0.26 |
| retentate | | | | |
| Third | 3861 | 99.76 | 0.20 | 0.04 |
| permeate | | | | |
| Fourth | 391 | 55.35 | 44.08 | 0.57 |
| retentate | | | | |
| Fourth | 1000 | 98.84 | 1.02 | 0.14 |
| permeate | | | | |

### Example 4

The calculation of example 1 was repeated for a raw gas containing 69.9% by volume of methane, 30.0% by volume of carbon dioxide and 0.1% by volume of oxygen. The optimization calculated a minimum for the recycle rate at 69.3% for a permeate side pressure of the first membrane separation unit of 3.10 bar and a distribution of 34.3 membrane modules in the first membrane separation unit, 183.7 membrane modules in the second membrane separation unit, 73.2 membrane modules in the third membrane separation unit and 38.8 membrane modules in the fourth membrane separation unit. The calculated flow rates and compositions of the process streams are given in table 7.

The calculation shows that the process of the invention can separate most of the carbon dioxide with a low methane content suitable for direct discharge to the atmosphere from a biogas with a high methane content, albeit with a higher recycle rate.

TABLE 7

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 30.0 | 69.9 | 0.1 |
| Feed | 16930 | 53.40 | 46.47 | 0.13 |
| First | 14039 | 44.46 | 55.39 | 0.14 |
| retentate | | | | |
| First | 2889 | 96.72 | 3.20 | 0.08 |
| permeate | | | | |
| Second | 7202 | 2.89 | 97.00 | 0.11 |
| retentate | | | | |
| Second | 6832 | 88.13 | 14.69 | 0.18 |
| permeate | | | | |
| Third | 645 | 86.14 | 13.64 | 0.22 |
| retentate | | | | |
| Third | 2244 | 99.76 | 0.20 | 0.04 |
| permeate | | | | |
| Fourth | 95 | 22.45 | 77.04 | 0.51 |
| retentate | | | | |
| Fourth | 550 | 97.11 | 2.72 | 0.18 |
| permeate | | | | |

### Comparative Example 3

The calculation of example 1 was repeated for a raw gas containing 84.9% by volume of methane, 15.0% by volume of carbon dioxide and 0.1% by volume of oxygen. The optimization calculated a minimum for the recycle rate at 79.7% for a permeate side pressure of the first membrane separation unit of 3.45 bar and a distribution of 19 membrane modules in the first membrane separation unit, 226 membrane modules in the second membrane separation unit, 21 membrane modules in the third membrane separation unit and 33 membrane modules in the fourth membrane separation unit. The calculated flow rates and compositions of the process streams are given in table 8.

The calculation shows that the recycling rate increases if the CO₂ content in the feed stream is reduced. Also, the methane content in the fourth permeate stream increases, which increases the costs for oxidative post-treatment.

TABLE 8

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 24.0 | 84.9 | 0.1 |
| Feed | 17966 | 46.80 | 53.07 | 0.13 |
| First | 16662 | 42.97 | 56.89 | 0.14 |
| retentate | | | | |
| First | 1305 | 95.66 | 4.25 | 0.09 |
| permeate | | | | |
| Second | 8735 | 2.91 | 96.99 | 0.10 |
| retentate | | | | |
| Second | 7927 | 87.13 | 12.70 | 0.17 |
| permeate | | | | |
| Third | 538 | 89.80 | 10.03 | 0.17 |
| retentate | | | | |
| Third | 767 | 99.76 | 0.20 | 0.04 |
| permeate | | | | |
| Fourth | 40 | 7.37 | 92.28 | 0.35 |
| retentate | | | | |
| Fourth | 497 | 96.46 | 3.38 | 0.16 |
| permeate | | | | |

### Example 5

The calculation of example 1 was repeated for a raw gas containing 39.9% by volume of methane, 60.0% by volume of carbon dioxide and 0.1% by volume of oxygen. The optimization calculated a minimum for the recycle rate at 35.4% for a permeate side pressure of the first membrane separation unit of 3.45 bar and a distribution of 87 membrane modules in the first membrane separation unit, 92 membrane modules in the second membrane separation unit, 147 membrane modules in the third membrane separation unit and 17 membrane modules in the fourth membrane separation unit. The calculated flow rates and compositions of the process streams are given in table 9.

The calculation shows that the process of the invention can separate most of the carbon dioxide with a low methane content in the third permeate stream, suitable for direct discharge to the atmosphere from a biogas with a high low methane content. The recycling rate is very low.

TABLE 9

| | Flow | Carbon dioxide | Methane | Oxygen |
|---|---|---|---|---|
| | rate | concentration | concentration | concentration |
| Gas stream | [Nm³/h] | [% by volume] | [% by volume] | [% by volume] |
| Raw gas | 10000 | 60.0 | 39.9 | 0.1 |
| Feed | 13540 | 65.60 | 34.23 | 0.17 |
| First | 6517 | 32.22 | 67.55 | 0.23 |
| retentate | | | | |
| First | 7022 | 96.57 | 3.31 | 0.12 |
| permeate | | | | |
| Second | 4099 | 2.84 | 97.00 | 0.16 |
| retentate | | | | |
| Second | 2418 | 82.03 | 17.62 | 0.35 |
| permeate | | | | |
| Third | 1647 | 86.19 | 13.49 | 0.32 |
| retentate | | | | |
| Third | 5376 | 99.75 | 0.20 | 0.05 |
| permeate | | | | |
| Fourth | 1121 | 80.07 | 19.51 | 0.42 |
| retentate | | | | |
| Fourth | 525 | 99.27 | 0.61 | 0.12 |
| permeate | | | | |

### Example 6

Gas separation was calculated for separating a raw gas stream provided at 1.01 bar with a flow rate of 10,000 Nm³/h and containing 50.0% by volume of methane, 49.7% by volume of carbon dioxide, 0.2% by volume of nitrogen and 0.1% by volume with SEPURAN® Green membrane modules containing the same membranes as in example 1 and having a separation capacity of 2.460 mol s⁻¹ MPa⁻¹. Separation was calculated for a facility with 137 membrane modules in the first membrane separation unit, 83 membrane modules in the second membrane separation unit, 62 membrane modules in the third membrane separation unit and 85 membrane modules in the fourth membrane separation unit. The temperature dependency of permeation and the pressure drop within a module were accounted for by applying the equations known from M. Scholz et. al, Ind. Eng. Chem. Res. 52 (2013) 1079-1088. Feed temperature was set to 25° C., pressure on the retentate side of the second membrane separation unit was set to 16.0 bar and pressure on the retentate side of the fourth membrane separation unit was set to 3.20 bar. The calculated flow rates, pressures, temperatures and compositions of the process streams are given in table 10.

The calculation shows that almost half of the carbon dioxide contained in the raw gas can be separated as a gas stream containing only 0.3% by volume of methane at a recycle rate of only 28%.

TABLE 10

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 10000 | 1.01 | | 49.70 | 50.00 | 0.20 | 0.10 |
| Feed | 12792 | 16.08 | 25.0 | 54.10 | 45.54 | 0.20 | 0.16 |
| First | 6568 | 16.04 | 18.3 | 18.13 | 81.36 | 0.34 | 0.17 |
| retentate | | | | | | | |
| First | 6224 | 3.60 | 20.8 | 92.05 | 7.74 | 0.05 | 0.16 |
| permeate | | | | | | | |
| Second | 5156 | 16.00 | 15.6 | 3.02 | 96.48 | 0.38 | 0.12 |
| retentate | | | | | | | |
| Second | 1412 | 1.01 | 16.0 | 73.33 | 26.14 | 0.17 | 0.36 |
| permeate | | | | | | | |
| Third | 3823 | 3.31 | 19.7 | 87.28 | 12.41 | 0.08 | 0.22 |
| retentate | | | | | | | |
| Third | 2401 | 1.01 | 20.2 | 99.64 | 0.30 | 0 | 0.05 |
| permeate | | | | | | | |
| Fourth | 1380 | 3.20 | 17.1 | 66.29 | 33.08 | 0.21 | 0.43 |
| retentate | | | | | | | |
| Fourth | 2443 | 1.01 | 19.1 | 99.14 | 0.75 | 0.01 | 0.10 |
| permeate | | | | | | | |

### Example 7

The calculation of example 6 was repeated for a 5% lower flow rate raw gas stream of 9500 Nm³/h, reducing the pressure on the retentate side of the fourth membrane separation unit to maintain the same methane concentration of 0.3% by volume in the third permeate stream, which required reducing the pressure on the retentate side of the fourth membrane separation unit from 3.20 bar to 3.05 bar. The calculated flow rates, pressures, temperatures and compositions of the process streams are given in table 11.

The calculation shows that reducing the pressure on the retentate side of the fourth membrane separation unit can keep methane concentration in the third permeate stream at the target value when the flow rate of the raw gas stream decreases. However, this leads to an increase of the recycle rate from 28% to 30%.

TABLE 11

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 9500 | 1.01 | | 49.70 | 50.00 | 0.20 | 0.10 |
| Feed | 12381 | 16.08 | 25.0 | 54.57 | 45.06 | 0.20 | 0.17 |
| First | 6166 | 16.04 | 17.9 | 16.84 | 82.64 | 0.34 | 0.18 |
| retentate | | | | | | | |
| First | 6215 | 3.48 | 20.6 | 92.01 | 7.78 | 0.05 | 0.16 |
| permeate | | | | | | | |
| Second | 4881 | 16.00 | 15.3 | 2.65 | 96.85 | 0.38 | 0.12 |
| retentate | | | | | | | |
| Second | 1285 | 1.01 | 15.8 | 70.75 | 28.67 | 0.18 | 0.40 |
| permeate | | | | | | | |
| Third | 3944 | 3.17 | 19.7 | 87.61 | 12.09 | 0.08 | 0.22 |
| retentate | | | | | | | |
| Third | 2271 | 1.01 | 20.1 | 99.64 | 0.30 | 0 | 0.06 |
| permeate | | | | | | | |
| Fourth | 1596 | 3.05 | 17.6 | 70.56 | 28.86 | 0.19 | 0.40 |
| retentate | | | | | | | |
| Fourth | 2348 | 1.01 | 19.1 | 99.20 | 0.69 | 0.01 | 0.10 |
| permeate | | | | | | | |

### Example 8

The calculation of example 6 was repeated for a 5% lower flow rate raw gas stream of 9500 Nm³/h, reducing the temperature of the feed stream to maintain the same methane concentration of 0.3% by volume in the third permeate stream, which required reducing the temperature of the feed stream from 25° C. to 22.8° C. The calculated flow rates, pressures, temperatures and compositions of the process streams are given in table 12.

The calculation shows that reducing the temperature of the feed stream can keep methane concentration in the third permeate stream at the target value when the flow rate of the raw gas stream decreases. Recycle rate decreases from 28% to 26%.

TABLE 12

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 9500 | 1.01 | | 49.70 | 50.00 | 0.20 | 0.10 |
| Feed | 11960 | 16.08 | 22.8 | 53.38 | 46.25 | 0.20 | 0.16 |
| First | 6170 | 16.04 | 16.0 | 17.28 | 82.21 | 0.34 | 0.17 |
| retentate | | | | | | | |
| First | 5790 | 3.57 | 18.6 | 91.86 | 7.93 | 0.05 | 0.16 |
| permeate | | | | | | | |
| Second | 4887 | 16.00 | 13.3 | 2.83 | 96.67 | 0.38 | 0.12 |
| retentate | | | | | | | |
| Second | 1283 | 1.01 | 13.8 | 72.35 | 27.10 | 0.17 | 0.37 |
| permeate | | | | | | | |
| Third | 3475 | 3.30 | 17.5 | 86.67 | 13.02 | 0.09 | 0.23 |
| retentate | | | | | | | |
| Third | 2315 | 1.01 | 18.1 | 99.64 | 0.30 | 0 | 0.05 |
| permeate | | | | | | | |
| Fourth | 1177 | 3.20 | 14.6 | 62.45 | 36.86 | 0.24 | 0.45 |
| retentate | | | | | | | |
| Fourth | 2298 | 1.01 | 16.8 | 99.08 | 0.81 | 0.01 | 0.11 |
| permeate | | | | | | | |

### Example 9

The calculation of example 6 was repeated reducing the temperature of the first permeate stream instead of reducing the temperature of the feed stream. The temperature of the first permeate stream had to be reduced from 20.8° C. to 17.5° C. before feeding it to the third membrane separation unit to maintain the same methane concentration of 0.3% by volume in the third permeate stream. The calculated flow rates, pressures, temperatures and compositions of the process streams are given in table 13.

The calculation shows that reducing the temperature of the first permeate stream can keep methane concentration in the third permeate stream at the target value when the flow rate of the raw gas stream decreases without changing the recycle rate.

TABLE 13

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 9500 | 1.01 | | 49.70 | 50.00 | 0.20 | 0.10 |
| Feed | 12159 | 16.08 | 25.0 | 53.72 | 45.91 | 0.20 | 0.17 |
| First | 6188 | 16.04 | 18.2 | 17.13 | 82.35 | 0.34 | 0.17 |
| retentate | | | | | | | |
| First | 5970 | 3.58 | 20.8 | 91.64 | 8.15 | 0.05 | 0.16 |
| permeate | | | | | | | |
| Second | 4880 | 16.00 | 15.5 | 2.67 | 96.83 | 0.38 | 0.12 |
| retentate | | | | | | | |
| Second | 1308 | 1.01 | 16.0 | 71.11 | 28.33 | 0.18 | 0.39 |
| permeate | | | | | | | |
| Third | 3673 | 3.30 | 16.4 | 86.63 | 13.06 | 0.09 | 0.23 |
| retentate | | | | | | | |
| Third | 2298 | 1.01 | 16.9 | 99.64 | 0.30 | 0 | 0.06 |
| permeate | | | | | | | |
| Fourth | 1351 | 3.20 | 13.7 | 65.13 | 34.21 | 0.22 | 0.44 |
| retentate | | | | | | | |
| Fourth | 2322 | 1.01 | 15.8 | 99.14 | 0.75 | 0.01 | 0.11 |
| permeate | | | | | | | |

### Example 10

The calculation of example 6 was repeated for a raw gas stream having a higher methane concentration of 51.0% by volume and a lower carbon dioxide concentration of 48.7% by volume, reducing the pressure on the retentate side of the fourth membrane separation unit to maintain the same methane concentration of 0.3% by volume in the third permeate stream, which required reducing the pressure on the retentate side of the fourth membrane separation unit from 3.20 bar to 3.12 bar and caused by this measure decreasing the permeate side pressure of the first membrane separation unit (2) from 3.6 bar in Example 6 to 3.54 bar in Example 10. The calculated flow rates, pressures, temperatures and compositions of the process streams are given in table 14.

If, based on Example 6, the CH₄ concentration in the raw gas is increased by 1% point without adjusting the permeate side pressure of the first membrane separation unit (2), the CH₄ concentration in the permeate of the 3rd membrane separation unit (4) would increase from 0.30% to 0.32%. By lowering the permeate side pressure of the first membrane separation unit (2), in this example via reducing the pressure on the retentate side of the fourth membrane separation unit, a stable methane concentration of 0.30% in the third permeate stream can be achieved.

The calculation shows that reducing the pressure on the retentate side of the fourth membrane separation unit can keep methane concentration in the third permeate stream at the target value when the methane concentration in the raw gas stream increases. However, this leads to an increase of the recycle rate from 28% to 29%.

TABLE 14

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 10000 | 1.01 | | 48.70 | 51.00 | 0.20 | 0.10 |
| Feed | 12919 | 16.09 | 25.0 | 53.73 | 45.91 | 0.20 | 0.16 |
| First | 6675 | 16.04 | 18.3 | 17.91 | 81.59 | 0.33 | 0.17 |
| retentate | | | | | | | |
| First | 6244 | 3.54 5 | 20.8 | 92.03 | 7.77 | 0.05 | 0.16 |
| permeate | | | | | | | |
| Second | 5264 | 16.00 | 15.7 | 3.08 | 96.43 | 0.38 | 0.12 |
| retentate | | | | | | | |
| Second | 1411 | 1.01 | 16.1 | 73.25 | 26.22 | 0.17 | 0.36 |
| permeate | | | | | | | |
| Third | 3909 | 3.24 | 19.8 | 87.48 | 12.23 | 0.08 | 0.22 |
| retentate | | | | | | | |
| Third | 2335 | 1.01 | 20.3 | 99.64 | 0.30 | 0 | 0.05 |
| permeate | | | | | | | |
| Fourth | 1508 | 3.12 | 17.5 | 68.85 | 30.66 | 0.19 | 0.40 |
| retentate | | | | | | | |
| Fourth | 2401 | 1.01 | 19.2 | 99.18 | 0.71 | 0.01 | 0.10 |
| permeate | | | | | | | |

### Example 11

The calculation of example 6 was repeated for a raw gas stream having a higher methane concentration of 51.0% by volume and a lower carbon dioxide concentration of 48.7% by volume, reducing the temperature of the feed stream to maintain the same methane concentration of 0.3% by volume in the third permeate stream, which required reducing the temperature of the feed stream from 25° C. to 23.8° C. The calculated flow rates, pressures, temperatures and compositions of the process streams are given in table 15.

The calculation shows that reducing the temperature of the feed stream can keep methane concentration in the third permeate stream at the target value when the methane concentration in the raw gas stream increases. Recycle rate decreases from 28% to 27%.

TABLE 15

| | Flow | | | Concentration | | | |
|---|---|---|---|---|---|---|---|
| | rate | Pressure | Temperature | [% by volume] | | | |
| Gas stream | [Nm³/h] | [bar] | [° C.] | CO₂ | Methane | Nitrogen | Oxygen |
| Raw gas | 10000 | 1.01 | | 48.70 | 51.00 | 0.20 | 0.10 |
| Feed | 12692 | 16.08 | 23.8 | 53.12 | 46.52 | 0.20 | 0.16 |
| First | 6678 | 16.04 | 17.3 | 18.14 | 81.36 | 0.33 | 0.17 |
| retentate | | | | | | | |
| First | 6015 | 3.59 | 19.7 | 91.96 | 7.84 | 0.05 | 0.15 |
| permeate | | | | | | | |
| Second | 5268 | 16.00 | 14.6 | 3.18 | 96.33 | 0.38 | 0.12 |
| retentate | | | | | | | |
| Second | 1410 | 1.01 | 15.0 | 74.06 | 25.43 | 0.16 | 0.35 |
| permeate | | | | | | | |
| Third | 3656 | 3.30 | 18.6 | 87.00 | 12.70 | 0.08 | 0.22 |
| retentate | | | | | | | |
| Third | 2358 | 1.01 | 19.1 | 99.64 | 0.30 | 0 | 0.05 |
| permeate | | | | | | | |
| Fourth | 1283 | 3.20 | 15.8 | 64.57 | 34.79 | 0.22 | 0.43 |
| retentate | | | | | | | |
| Fourth | 2374 | 1.01 | 17.9 | 99.16 | 0.77 | 0.01 | 0.11 |
| permeate | | | | | | | |

### LIST OF REFERENCE SIGNS

1 compressor
2 first membrane separation unit
3 second membrane separation unit
4 third membrane separation unit
5 fourth membrane separation unit
6 methane oxidation unit
7 raw gas conduit
8 feed conduit
9 first retentate conduit
10 second retentate conduit
11 first permeate conduit
12 third retentate conduit
13 fourth retentate conduit
14 second permeate conduit
15 third permeate conduit
16 fourth permeate conduit
17 pressure regulating valve
18 methane concentration sensor
19 heat exchanger
20 flow regulating valve
21 additional conduit
22 flow regulating valve

1-26. (canceled)

27. A facility for separating methane and carbon dioxide from a gas stream, the facility comprising:
a compressor (1);
four membrane separation units (2) to (5), each membrane separation unit comprising a gas separation membrane having higher permeance for carbon dioxide than for methane, a gas inlet, a retentate outlet and a permeate outlet;
a methane oxidation unit (6);
a raw gas conduit (7) connected to an inlet of the compressor (1);
a feed conduit (8) connecting an outlet of the compressor (1) with the gas inlet of the first membrane separation unit (2);
a first retentate conduit (9) connecting the retentate outlet of the first membrane separation unit (2) to the gas inlet of the second membrane separation unit (3);
a second retentate conduit (10) connected to the retentate outlet of the second membrane separation unit (3);
a first permeate conduit (11) connecting the permeate outlet of the first membrane separation unit (2) to the gas inlet of the third membrane separation unit (4);
a third retentate conduit (12) connecting the retentate outlet of the third membrane separation unit (4) to the gas inlet of the fourth membrane separation unit (5);
a fourth retentate conduit (13) connecting the retentate outlet of the fourth membrane separation unit (5) to an inlet of the compressor (1);
a second permeate conduit (14) connecting the permeate outlet of the second membrane separation unit (3) to an inlet of the compressor (1);
a third permeate conduit (15) connected to the permeate outlet of the third membrane separation unit (4); and
a fourth permeate conduit (16) connected to the permeate outlet of the fourth membrane separation unit (5);
wherein:
the third permeate conduit (15) is configured to discharge the third permeate to the surrounding atmosphere;
the fourth permeate conduit (16) connects the permeate outlet of the fourth membrane separation unit (5) to the methane oxidation unit (6);
the first membrane separation unit (2) comprises a membrane with a pure gas selectivity for carbon dioxide over methane, determined at 20° C. and 5 bar, of at least 30;
the facility is configured to provide a carbon dioxide concentration in the gas stream in the first permeate conduit (11), the first permeate stream, in a range of from 90 to 99% by volume.

28. The facility of claim 27, wherein:
the permeate side pressure in the first membrane separation unit (2) and the separation capacities, which are the product of the membrane area and the membrane permeance for carbon dioxide at a temperature of 25° C. and a feed side pressure of 5 bar, in the four membrane separation units (2) to (5) are configured to provide a carbon dioxide concentration in the first permeate stream of from 90 to 99% by volume;
and/or
the facility comprises means for controlling the permeate side pressure in the first membrane separation unit (2) and/or the separation capacities in the four membrane separation units (2) to (5) to provide a carbon dioxide concentration in the first permeate stream of from 90 to 99% by volume.

29. The facility of claim 27, wherein the methane oxidation unit (6) comprises a catalytic oxidizer, a regenerative thermal oxidizer or a biofilter.

30. The facility of claim 27, wherein the first permeate conduit (11) connects the permeate outlet of the first membrane separation unit (2) to the gas inlet of the third membrane separation unit (4) without any intermediary compressor or pump.

31. The facility of claim 27, wherein the separation capacity of the second membrane separation unit (3) is larger than the separation capacity of the fourth membrane separation unit (5), the separation capacity of a membrane separation unit being the product of the membrane area of the membrane separation unit and the membrane permeance for carbon dioxide at 25° C. and a feed side pressure of 5 bar.

32. The facility of claim 27, wherein a pressure regulating valve (17) is arranged in the fourth retentate conduit (13).

33. The facility of claim 27, wherein a methane concentration sensor (18) is connected to the third permeate conduit (15).

34. The facility of claim 33, comprising a pressure regulating valve (17) arranged in the fourth retentate conduit (13) and a controller controlling the pressure regulating valve (17) based on data measured by the methane concentration sensor (18).

35. The facility of claim 33, comprising a heat exchanger (19) in the feed conduit (8), a flow regulating valve (20) controlling flow of a heating or cooling fluid to the heat exchanger (19) and a controller controlling this flow regulating valve (20) based on data measured by the methane concentration sensor (18).

36. The facility of claim 33, wherein the third membrane separation unit (4) comprises a multitude of membrane modules arranged in parallel, at least one of said membrane modules comprising shut-off valves blocking flow through the membrane module, and a controller controlling the shut-off valves based on data measured by the methane concentration sensor (18).

37. The facility of claim 33, wherein the first membrane separation unit (2) comprises a bore-side fed hollow fiber membrane module with the gas inlet on a first end of the module, the retentate outlet on a second end of the module opposite to the first end, the first permeate outlet adjacent to the first end of the module and connected to the first permeate conduit (11) and an additional permeate outlet adjacent to the second end of the module; the facility further comprising an additional conduit (21) connecting the additional permeate outlet with the gas inlet of the fourth membrane separation unit (5), a flow regulating valve (22) arranged in the additional conduit (21) and a controller controlling this flow regulating valve (22) based on data measured by the methane concentration sensor (18).

38. A membrane process for separating methane and carbon dioxide from a gas stream, comprising:
(a) providing the facility of claim 27;
(b) introducing a raw gas stream, containing from 20 to 60% by volume, carbon dioxide and having a combined content of methane and carbon dioxide of at least 95% by volume, into the raw gas conduit (7) of said facility;
(c) compressing the raw gas stream combined with recycle streams from the fourth retentate conduit (13) and the second permeate conduit (14) with compressor (1) to provide a feed stream at a feed pressure of from 7 to 25 bar and a temperature of from 15 to 50° C.;
(d) separating the feed stream in the first membrane separation unit (2) into a first permeate stream and a first retentate stream, using a membrane with a mixed gas selectivity for carbon dioxide over methane of at least 30, at the feed pressure and the temperature of the feed stream, and selecting permeate side pressure in the first membrane separation unit and separation capacities in the four membrane separation units to provide a carbon dioxide concentration in the first permeate stream of from 90 to 99% by volume, the separation capacity of a membrane separation unit being the product of the membrane area and the membrane permeance for carbon dioxide at a temperature of 25° C. and a feed side pressure of 5 bar;
(e) separating the first retentate stream in the second membrane separation unit (3) into a second retentate stream and a second permeate stream, further processing the second retentate stream or withdrawing the second retentate stream as a methane rich product stream and recycling the second permeate stream through the second permeate conduit (14);
(f) separating the first permeate stream in the third membrane separation unit (4) into a third retentate stream and a third permeate stream, discharging the third permeate stream to the surrounding atmosphere without further methane removal;
(g) separating the third retentate stream in the fourth membrane separation unit (5) into a fourth retentate stream and a fourth permeate stream, recycling the fourth retentate stream through the retentate conduit (13); and
(h) oxidizing the fourth permeate stream in the methane oxidation unit (6) to provide an off-gas stream containing less than 0.3% by volume methane, which off-gas stream is discharged to the surrounding atmosphere.

39. The process of claim 38, wherein the concentration of methane in the third permeate stream is measured with a methane concentration sensor (18) and an operating parameter of the first membrane separation unit (2) is adjusted based on the measured value to maintain the concentration of methane in the third permeate stream at or below a target value.

40. The process of claim 39, wherein the permeate side pressure of the first membrane separation unit (2) is adjusted based on the measured concentration of methane in the third permeate stream, decreasing the permeate side pressure when the concentration of methane in the third permeate stream rises to above the target value.

41. The process of claim 40, wherein the permeate side pressure of the first membrane separation unit (2) is controlled with a pressure regulating valve (17) arranged in the fourth retentate conduit (13).

42. The process of claim 39, wherein the temperature of the feed stream is adjusted based on the measured concentration of methane in the third permeate stream, decreasing the temperature of the feed stream when the concentration of methane in the third permeate stream rises to above the target value.

43. The process of claim 38, wherein the temperature of the first permeate stream is adjusted based on the measured concentration of methane in the third permeate stream, decreasing the temperature of the first permeate stream when the concentration of methane in the third permeate stream rises to above the target value.

44. The process of claim 38, wherein the separation capacity of the second membrane separation unit (3) is selected to provide a carbon dioxide concentration in the second retentate stream of from 0.5 to 4.0% by volume and the separation capacity of the fourth membrane separation unit (5) is selected to provide a methane recovery with the second retentate stream of from 98.0 to 99.9%.

45. The process of claim 38, wherein the feed pressure and the permeate side pressure of the first membrane separation unit (2) are selected to provide a pressure ratio in the third membrane separation unit (4) which is from 0.4 to 1.0 times the pressure ratio in the first membrane separation unit (2), the pressure ratio in a membrane unit being the ratio between the feed side pressure and the permeate side pressure in the membrane unit.

46. The process of claim 38, wherein the methane oxidation unit (6) comprises a catalytic oxidizer or a regenerative thermal oxidizer and the separation capacity of the fourth membrane separation unit is selected to provide a methane concentration in the fourth permeate stream which allows autothermal operation of the oxidizer.
